(19) United States
(12) Patent Application Publication
(10) Pub. No.: US 2025/0196209 A1
(43) Pub. Date: Jun. 19, 2025

(54) METHOD, SYSTEM AND APPARATUS FOR DETERMINING POLLUTANT CONTROL STRATEGY, AND COMPUTER DEVICE

(71) Applicant: TSINGHUA UNIVERSITY, BEIJING (CN)

(72) Inventor: Deyi HOU, BEIJING (CN)

(21) Appl. No.: 18/762,677

(22) Filed: Jul. 3, 2024

Related U.S. Application Data
(63) Continuation of application No. PCT/CN2024/075763, filed on Feb. 4, 2024.

(30) Foreign Application Priority Data
Dec. 18, 2023 (CN) 202311744915.9

(51) Int. Cl. B09C 1/00; C02F 1/00; C02F 103/06; G01N 33/18

(52) U.S. Cl. CPC B09C 1/002; C02F 1/008; G01N 33/18; B09C 2101/00; C02F 2103/06; C02F 2209/006; C02F 2209/03; C02F 2209/40

(57) ABSTRACT

A method, a system and an apparatus for determining pollutant control strategy, and a computer device. The method includes: obtaining water body data of different locations of a polluted water body and historical sample water body data of different pollution levels, and identifying pollutant distribution information; collecting water flow information of the polluted water body, and generating a pollutant control strategy for the polluted water body; executing the pollutant control strategy, and detecting change information of the water body data of each location; adjusting the pollutant control strategy for the polluted water body to obtain a new pollutant control strategy, replacing the pollutant control strategy with the new pollutant control strategy, iteratively executing steps of executing the pollutant control strategy and detecting the change information of the water body data of each of the different locations, until current water body data of each location meet a standard water body condition.

### CROSS-REFERENCE TO RELATED APPLICATIONS

The present application is a continuation application of PCT Patent Application No. PCT/CN2024/075763, entitled “Method, System and Apparatus for Determining Pollutant Control Strategy, and Computer Device”, filed on Feb. 4, 2024, which claims the priority of the Chinese patent application No. 202311744915.9, filed on Dec. 18, 2023 and entitled “Method, System and Apparatus for Determining Pollutant Control Strategy, and Computer Device”, the entire contents of which are incorporated herein by reference.

### TECHNICAL FIELD

The present application relates to the technical field of risk management and control of polluted sites, and in particular to methods, systems and apparatuses for determining pollutant control strategy, and computer devices.

### BACK GROUND

Because groundwater pollution is hidden, long-term, and difficult to recover, the groundwater pollutions of the sites caused by industrial activities such as chemical production, waste treatment, metal smelting, etc., are difficult to detect and have high recovery costs, which has become an environmental problem currently faced. The pollutants generally have characteristics of biological toxicity, environmental persistence, and bioaccumulation, thus posing a serious threat to the groundwater environment and human's health. Therefore, how to intelligently manage and control the pollutants in the groundwater pollutions is a current research focus.

An existing intelligent manage and control method for the pollutants in the groundwater pollutions is monitoring current pollution situations of the groundwater, and then performing an intelligent manage and control for pollutants in groundwater pollution by predicting a pollution trend prediction model of the groundwater. However, the pollution trend prediction model is often a model generated based on historical water body data, and the prediction accuracies for different water bodies are different. When an intelligent manage and control is carried out based on the pollution trend prediction model, a pollution plume leakage may arise easily, thus causing the effects of the manage and control for the pollutants in different groundwater pollutions to be poor.

### SUMMARY

In view of the technical problems above, it is necessary to provide methods, systems and apparatuses for determining pollutant control strategy, computer devices, non-transitory computer-readable storage mediums, and computer program products.

In a first aspect, the present application provides a method for determining a pollutant control strategy. The method includes:
obtaining water body data of different locations of a polluted water body and historical sample water body data of different pollution levels, and identifying pollutant distribution information of the polluted water body based on the historical sample water body data and the water body data of the different locations;
collecting water flow information of the polluted water body, and generating a pollutant control strategy for the polluted water body based on the water flow information of the polluted water body and the pollutant distribution information of the polluted water body;
executing the pollutant control strategy, and detecting change information of the water body data of each of the different locations; and
adjusting the pollutant control strategy for the polluted water body based on the change information of the water body data of each of the different locations to obtain a new pollutant control strategy, replacing the pollutant control strategy with the new pollutant control strategy, iteratively executing steps of executing the pollutant control strategy and detecting the change information of the water body data of each of the different locations, until current water body data of each of the different locations meet a standard water body condition.

In an embodiment of the present application, identifying the pollutant distribution information of the polluted water body based on the historical sample water body data and the water body data of the different locations, includes:
identifying target historical sample water body data corresponding to the water body data of each of the different locations from the historical sample water body data, and using a pollution level of each of the target historical sample water body data as a pollution level of the water body data of each corresponding location; and
identifying relative position information between the different locations, and generating the pollutant distribution information of the polluted water body based on the relative position information and pollution levels of the water body data of the different locations.

In an embodiment of the present application, generating the pollutant control strategy for the polluted water body based on the water flow information of the polluted water body and the pollutant distribution information of the polluted water body, includes:
identifying a hydraulic gradient of the polluted water body and a water flow direction of the polluted water body based on the water flow information, and identifying a diffusion direction of the polluted water body and a diffusion rate of the polluted water body, based on the hydraulic gradient of the polluted water body and the water flow direction of the polluted water body and the pollutant distribution information of the polluted water body;
dividing the polluted water body into different polluted regions based on the identified diffusion directions of the polluted water body and the identified diffusion rates of the polluted water body to obtain pollution diffusion information corresponding to different polluted regions, and identifying a pollution level corresponding to each of the different polluted regions; and
querying a pollution control sub-strategy for each of the different polluted regions from a pollution-treatment strategy database, based on the pollution level corresponding to each of the different polluted regions and the pollution diffusion information corresponding to each of the different polluted regions, and using pollution control sub-strategies for the different polluted regions as the pollutant control strategy for the polluted water body.

In an embodiment of the present application, adjusting the pollutant control strategy for the polluted water body based on the change information of the water body data of each of the different locations to obtain a new pollutant control strategy, includes:
identifying current pollutant distribution information of the polluted water body based on the change information of the water body data of each of the different locations, and returning to execute collecting the water flow information of the polluted water body to obtain a new pollution control sub-strategy corresponding to each new polluted region of the polluted water body; and
using new pollution control sub-strategies for all new polluted regions as the new pollutant control strategy for the polluted water body.

In an embodiment of the present application, iteratively executing the steps of executing the pollutant control strategy and detecting the change information of the water body data of each of the different locations, until the current water body data of each of the different locations meet the standard water body condition, includes:
collecting current water body data of each of the different locations, and identifying first historical sample water body data corresponding to the current water body data of each of the different locations from the historical sample water body data, and using a pollution level of the first historical sample water body data as a pollution level corresponding to the current water body data of each of the different locations;
judging whether there is a pollution level greater than a pollution level threshold; and
ending iteratively executing the steps of executing the pollutant control strategy and detecting the change information of the water body data of each of the different locations if there is no pollution level greater than the pollution level threshold.

In an embodiment of the present application, the pollutant distribution information of the polluted water body is used to characterize water body regions of the polluted water body corresponding to different pollution levels of the polluted water body.

In an embodiment of the present application, the hydraulic gradient is gradient distribution information of water flow pressures at the different locations.

In an embodiment of the present application, generating the pollutant distribution information of the polluted water body based on the relative position information and the pollution levels of the water body data of the different locations, includes: identifying position information of a pollution center point of the polluted water body through a two-dimensional plane image recognition strategy based on the pollution levels of the different locations and the relative position information between the different locations; and constructing a circular range of each of the pollution levels of the polluted water body by taking the pollution center point as a center of a circle and taking a straight-line distance between each of the different locations and the pollution center point as a radius, and obtaining the pollutant distribution information of the polluted water body.

In an embodiment of the present application, dividing the polluted water body into the different polluted regions based on the diffusion directions of the polluted water body and the diffusion rates of the polluted water body, includes: regarding regions, which have the same diffusion direction, and whose diffusion-rate deviation values each are not greater than a preset deviation threshold, as one polluted region.

In an embodiment of the present application, each polluted region includes one or more pollution levels corresponding to the polluted regions, and corresponding to the polluted region includes multiple pollution levels, the highest pollution level among the multiple pollution levels is used as the pollution level corresponding to the polluted region.

In an embodiment of the present application, a boundary of the pollutant distribution information of the polluted water body includes a boundary of a circular range of each pollution level located in a water flow direction, and a shore boundary located in a direction perpendicular to the water flow direction.

In a second aspect of the present application, a system for determining pollutant control strategy is provided, and the system includes a head pressure detecting subsystem, a groundwater extracting subsystem, a groundwater directional injecting subsystem, a flow detecting subsystem, a pollutant monitoring subsystem, and a control center subsystem.

The control center subsystem is connected to the head pressure detecting subsystem, the groundwater extracting subsystem, the groundwater directional injecting subsystem, the flow detecting subsystem, and the pollutant monitoring subsystem respectively.

The head pressure detecting subsystem is connected to the groundwater extracting subsystem and the groundwater directional injecting subsystem, respectively.

The flow detecting subsystem is configured to collect water flow information of a polluted water body, and transmit the water flow information of the polluted water body to the control center subsystem.

The pollutant monitoring subsystem is configured to collect water body data of different locations of the polluted water body, and transmit the water body data of the different locations of the polluted water body to the control center subsystem.

The groundwater extracting subsystem and the groundwater directional injecting subsystem are configured to receive pollutant control instructions of executing the pollutant control strategy transmitted by the control center subsystem, and configured to execute the pollutant control strategy according to the pollutant control instructions.

The head pressure detecting subsystem is configured to detect execution information of the groundwater extracting subsystem and the groundwater directional injecting subsystem responding to the pollutant control instructions, and operation information of the groundwater extracting subsystem and the groundwater directional injecting subsystem, and configured to transmit the execution information and the operation information of the groundwater extracting subsystem and the groundwater directional injecting subsystem to the control center subsystem.

The control center subsystem is configured to execute any one of the methods for determining pollutant control strategy above.

In an embodiment of the present application, the groundwater directional injecting subsystem includes a vacuum extraction subsystem and an injection pump, and the groundwater extracting subsystem includes a suction pump.

The injection pump of the groundwater directional injecting subsystem, the suction pump of the groundwater extracting subsystem, and the vacuum extraction subsystem are arranged in the same extraction well.

The groundwater directional injecting subsystem is configured to identify an extraction well corresponding to an injection well and an extraction well corresponding to a vacuum extraction capture well from all extraction wells based on the pollutant control instructions, and simultaneously start an injection pump in the extraction well corresponding to the injection well and a vacuum pumping device of the extraction well corresponding to the vacuum extraction capture well.

The groundwater extracting subsystem is configured to identify an extraction well corresponding to a pumping well from all extraction wells based on the pollutant control instructions, and start a suction pump of the extraction well corresponding to the pumping well.

The head pressure detecting subsystem comprises a water flow pressure sensor and a flow direction sensor. The flow detecting subsystem comprises a water flow pressure sensor. The water flow pressure sensor and the flow direction sensor of the head pressure detecting subsystem, and the water flow pressure sensor of the flow detecting subsystem are arranged in the same extraction well.

In an embodiment of the present application, the injection pump is configured to inject high-pressure water into the polluted water body; the vacuum pumping device is configured to perform a vacuum extraction operation on the polluted water body to create a low-pressure zone, and a hydraulic communication channel is constructed based on the low-pressure zone and the high-pressure water, and is configured to guide the high-pressure water into the polluted water body.

Corresponding to an extraction well being an injection well, the extraction well opens directing mesh holes arranged therein, and the directing mesh holes are configured to guide injection directions of the high-pressure water. A hole spacing between the directing mesh holes is less than a preset hole spacing threshold. Orientations of the directing mesh holes are determined by a direction of the extraction well corresponding to the vacuum extraction capture well relative to the extraction well corresponding to the injection well.

Each extraction well comprises a first well section exposed to air and a second well section in the water body. Based on a height H₁ of a vadose zone in the first well section and a thickness H₂ of an aquifer corresponding to the second well section, a pressure P of a high-pressure water injection of the injection pump and a pumping pressure P′ of the suction pump are determined. The pressure P of the high-pressure water injection satisfies P>ρg(1.7H₁+H₂), and the pumping pressure P′ forms a drawdown greater than H₂/2.

In an embodiment of the present application, the preset hole spacing threshold is 5 cm, or the directing mesh holes are wrapped with a protective net outside.

In an embodiment of the present application, the number of extraction wells is greater than three.

In a third aspect of the present application, an apparatus for determining pollutant control strategy is provided, and the apparatus includes an obtaining circuit, a generating circuit, a detection circuit, and an iteration circuit.

The obtaining circuit is configured to obtain water body data of different locations of a polluted water body and historical sample water body data of different pollution levels, and identify pollutant distribution information of the polluted water body based on the historical sample water body data and the water body data of the different locations.

The generating circuit is configured to collect water flow information of the polluted water body, and generate a pollutant control strategy for the polluted water body based on the water flow information of the polluted water body and the pollutant distribution information of the polluted water body.

The detection circuit is configured to execute the pollutant control strategy, and detect change information of the water body data of each of the different locations.

The iteration circuit is configured to adjust the pollutant control strategy for the polluted water body based on the change information of the water body data of each of the different locations to obtain a new pollutant control strategy, configured to replace the pollutant control strategy with the new pollutant control strategy, configured to iteratively execute the steps of executing the pollutant control strategy and detecting the change information of the water body data of each of the different locations until current water body data of each of the different locations meet a standard water body condition.

In a fourth aspect of the present application, a computer device is provided. The computer device includes a processor, and a memory having computer programs stored thereon. The processor, when executing the computer programs, performs steps of any one of the methods in the first aspect.

In a fifth aspect of the present application, a non-transitory computer-readable storage medium, having computer programs stored thereon, is provided. The computer programs, when executed by a processor, cause the processor to perform steps of any one of the methods in the first aspect.

In a sixth aspect of the present application, a computer program product, comprising executable instructions, is provided. The executable instructions, when executed by a processor, cause the processor to perform steps of any one of the methods in the first aspect.

In the method, system, apparatus for determining the pollutant control strategy, and the computer device above, the water body data of different locations of a polluted water body and the historical sample water body data of different pollution levels are obtained, and the pollutant distribution information of the polluted water body is identified based on the historical sample water body data and the water body data of different locations. The water flow information of the polluted water body is collected, and the pollutant control strategy for the polluted water body is generated based on the water flow information of the polluted water body and the pollutant distribution information of the polluted water body. The pollutant control strategy is executed, and change information of the water body data of each of the different locations is detected. The pollutant control strategy for the polluted water body is adjusted based on the change information of the water body data of each of the different locations, to obtain a new pollutant control strategy, and the pollutant control strategy is replaced by the new pollutant control strategy, and the steps of executing the pollutant control strategy and detecting the change information of the water body data of each of the different locations are iteratively executed, until the current water body data of each of the different locations meet a standard water body condition. In the above solutions, by collecting water body data of different locations of the polluted water body, the pollutant distribution information of the polluted water body is analyzed, and then the pollutant control strategy are generated based on collected water flow information of the polluted water body and the pollutant distribution information of the polluted water body, and then the system for determining pollutant control strategy is controlled to execute the instructions corresponding to the instructions. Then the pollutant control strategy is adjusted in real time to ensure intelligent control of pollutant treatment of the polluted water body. In this solution, the pollutant control strategy for the polluted water body is generated in real time based on the pollutant distribution information and the water flow information of the polluted water body, and the pollutant control strategy is adjusted by monitoring current water body data of the polluted water body in real time, thereby improving the applicability of the intelligent pollutant treatment for different polluted water bodies, and improving the effects of pollutant management and control for different groundwater pollutions.

### BRIEF DESCRIPTION OF THE DRAWINGS

FIG. 1 is a view showing an application scenario of a method for determining pollutant control strategy according to an embodiment of the present application.

FIG. 2 is a schematic flow chart of a method for determining pollutant control strategy according to an embodiment of the present application.

FIG. 3 is a view showing a control center subsystem according to an embodiment of the present application.

FIG. 4 is a view showing execution of control instructions for pollutant according to an embodiment of the present application.

FIG. 5 is a view showing a groundwater directional injecting subsystem according to an embodiment of the present application.

FIG. 6 is a view showing directing mesh holes of an embodiment of the present application.

FIG. 7 is a view showing an extraction well according to an embodiment of the present application.

FIG. 8 is a schematic flow chart of a method for determining pollutant control strategy according to an embodiment of the present application.

FIG. 9 is a block diagram showing a structure of an apparatus for determining pollutant control strategy according to an embodiment of the present application.

FIG. 10 is a view showing an internal structure of a computer device according to an embodiment of the present application.

### DETAILED DESCRIPTION OF THE EMBODIMENTS

In order to make the objectives, technical solutions and advantages of the present application clearer and to be better understood, the present application is further described in detail in conjunction with the accompanying drawings and embodiments herein. It should be understood that the specific embodiments described herein are only used to explain the present application but not intended to limit the present application.

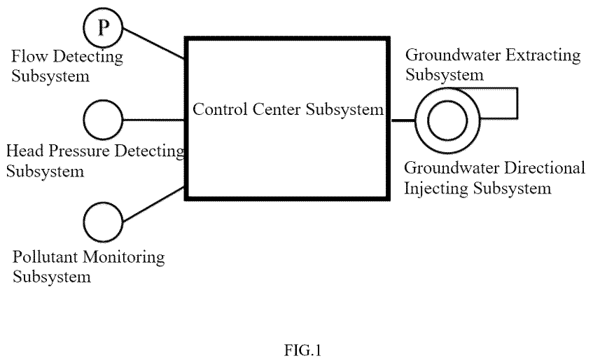

A method for determining pollutant control strategy provided by an embodiment of the present application may be applied to an application scenario of groundwater pollutant management and control as shown in FIG. 1. The method is applied to a system for determining pollutant control strategy, and the system includes a head pressure detecting subsystem, a groundwater extracting subsystem, a groundwater directional injecting subsystem, a flow detecting subsystem, a pollutant monitoring subsystem, and a control center subsystem. The control center subsystem is connected to the head pressure detecting subsystem, the groundwater extracting subsystem, the groundwater directional injecting subsystem, the flow detecting subsystem, and the pollutant monitoring subsystem respectively. The head pressure detecting subsystem is connected to the groundwater extracting subsystem and the groundwater directional injecting subsystem respectively. The method for determining pollutant control strategy is applied to the control center subsystem. The control center subsystem may be a terminal, and the terminal may be but is not limited to any personal computer, laptop, smart phone, or tablet computer, etc. The terminal analyzes pollutant distribution information of the polluted water body by collecting water body data of different locations of the polluted water body, and then, by collecting water flow information of the polluted water body and the pollutant distribution information of the polluted water body, generates a pollutant control strategy, and controls the system for determining pollutant control strategy to execute instructions corresponding to the pollutant control strategy, and finally adjusts the pollutant control strategy in real time to ensure an intelligent control of pollutant treatment for the polluted water body. In this solution, the pollutant control strategy for the polluted water body is generated in real time based on the pollutant distribution information and the water flow information of the polluted water body, and the pollutant control strategy is adjusted by detecting current water body data of the polluted water body in real time, thereby improving the applicability of intelligent controls of pollutant treatments for different polluted water bodies, and improving the effects of pollutant management and control for different groundwater pollutions.

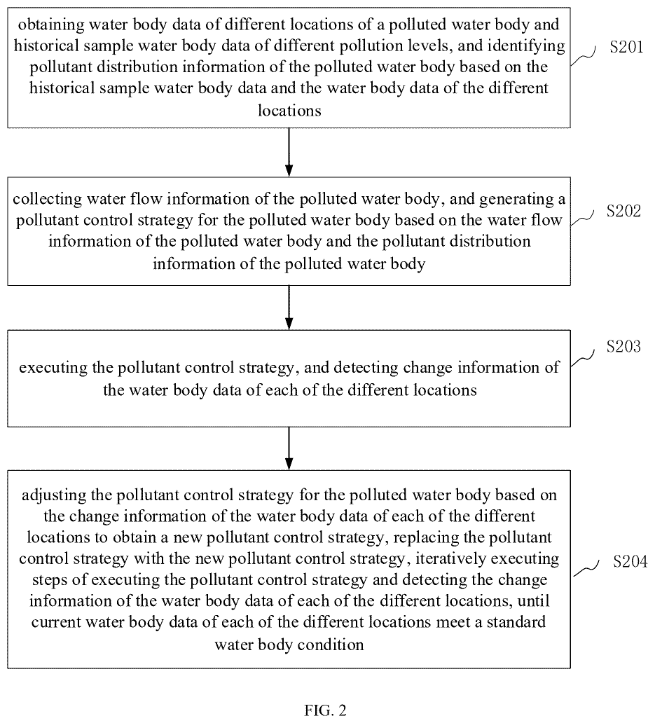

In an embodiment, as shown in FIG. 2, a method for determining pollutant control strategy is provided. The method includes the following steps S201 to S204 which will be described by taking the method applied to a terminal as an example.

In Step S201, water body data of different locations of a polluted water body and historical sample water body data of different pollution levels are obtained, and pollutant distribution information of the polluted water body is identified based on the historical sample water body data and the water body data of different locations.

In this embodiment, the terminal receives the water body data of different locations of the polluted water body transmitted by the pollutant monitoring subsystem. The water body data may include water quality parameters and pollutant concentrations. Conventional water quality parameters include but are not limited to pH, dissolved oxygen, temperature, conductivity, total dissolved solids (TDS), etc. The pollutant concentrations include concentrations of pollutants, such as heavy metals, organic matter, nitrogen, phosphorus, etc., which exceed a pollution level threshold. The pollutant monitoring subsystem includes samplers evenly arranged at different locations of the groundwater body, and the water body data of different locations of the polluted water body in the groundwater body are collected by the samplers at different locations. Then, the terminal selects sample water body data of different pollution levels from the historical water body database and uses them as the historical sample water body data of different pollution levels. Then, based on the historical sample water body data, the terminal identifies a sub-pollution level of the water body data of each location, obtaining the pollutant distribution information of the polluted water body. The pollutant distribution information is used to characterize water body regions of the polluted water body corresponding to the different pollution levels of the polluted water body. The specific identification process will be described in detail hereinafter. Sensors in the pollutant monitoring subsystem are placed at locations preset by a staff.

In Step S202, water flow information of the polluted water body is collected, and a pollutant control strategy for the polluted water body is generated based on the water flow information of the polluted water body and the pollutant distribution information of the polluted water body.

In this embodiment, the terminal receives the water flow information of the polluted water body transmitted by the flow detecting subsystem. The flow detecting subsystem includes water flow pressure sensors and flow direction sensors uniformly arranged at different positions in the groundwater body, and the water flow information of the polluted water body is determined based on the water flow pressure sensors and the flow direction sensors at different positions. The water flow information includes a hydraulic gradient of an aquifer of the groundwater and a water flow direction of the groundwater. The hydraulic gradient is gradient distribution information of the water flow pressures at different positions detected by the water flow sensors. Then, the terminal generates the pollutant control strategy for the polluted water body based on the water flow information of the polluted water body and the pollutant distribution information of the polluted water body. The pollutant control strategy includes pollutant control sub-strategies for different polluted regions of the polluted water body, and each pollutant control sub-strategy is used to avoid a spread of pollution plume in the polluted region, and used to treat the pollutants in the polluted region. The specific treatment process and the process of generating the pollutant control strategy for the polluted water body will be described in detail hereinafter. The pollution plume is an annular range surrounding all pollutants and formed by the polluted water body.

In Step S203, the pollutant control strategy is executed, and change information of the water body data of each of the different locations is detected.

In this embodiment, the terminal executes the pollutant control strategy through the groundwater extracting subsystem and the groundwater directional injecting subsystem. The staff constructs a plurality of extraction wells arranged in the groundwater area according to a fixed rule. The extraction wells are connected to pumping pipelines respectively, and then pumping pipelines are connected to the polluted-groundwater treatment system by a main pipe. Each groundwater extracting subsystem includes a suction pump arranged in each extraction well. The groundwater directional injecting subsystem includes an injection pump arranged in each extraction well. The suction pump is configured to extract pollutants from the polluted water body, thereby preventing the pollution plume of the polluted water body from spreading in the direction of the water flow. The injection pump is configured to inject high-pressure fluid into the polluted water body. The main component of the fluid is water, and the high-pressure water is used to prevent the pollution plume from continuing to spread in the direction of the water flow. The groundwater extracting subsystem and the groundwater directional injecting subsystem form a hydraulic communication channel in the groundwater through the suction pumps and the injection pumps. The groundwater extracting subsystem and the groundwater directional injecting subsystem select some of the extraction wells to serve as vacuum extraction capture wells based on the generated pollutant control strategy, thus performing a vacuum extraction while performing a high-pressure injection, so as to create a low-pressure zone to provide a direction guidance for the fluid injected under high pressure. The orientations of the mesh holes of a mesh tube of the groundwater directional injecting subsystem are determined by the position of the vacuum extraction capture well, so as to enhance the directionality of a high-pressure injection and ensure a successful construction of a hydraulic communication channel. The hydraulic communication channel is used to ensure that the pollution plume can shrink in the direction of the hydraulic communication channel, thereby avoiding the spread of the pollution plume. Then, the terminal, based on the current water body data of each location transmitted by the pollutant monitoring subsystem, identifies the change information between the current water body data and the water body data collected for the first time, thus obtaining the change information of the water body data.

In Step S204, the pollutant control strategy for the polluted water body is adjusted based on the change information of the water body data of each of the different locations, to obtain a new pollutant control strategy, and the pollutant control strategy is replaced by the new pollutant control strategy, and the steps of executing the pollutant control strategy and detecting the change information of the water body data of each of the different locations are iteratively executed, and the iterative execution does not end until the current water body data of each of the different locations meet a standard water body condition.

In this embodiment, the terminal adjusts the pollutant control strategy for the polluted water body based on the change information of the water body data of each location, obtaining a new pollutant control strategy. The specific adjustment process will be described in detail hereinafter. Then, the terminal replaces the pollutant control strategy with the new pollutant control strategy, iteratively executes the steps of executing the pollutant control strategy and detecting the change information of the water body data of each of the different locations, and the iterative execution does not end until the current water body data of each location meet the standard water body condition. The standard water body condition is that a pollution level of the current water body data of each location is less than a pollution level threshold preset in the terminal.

Based on the above solutions, water body data of different locations of the polluted water body is collected, the pollutant distribution information of the polluted water body is analyzed based on the collected water body data, and then the water flow information of the polluted water body is collected, a pollutant control strategy is generated based on collected water flow information of the polluted water body and the pollutant distribution information of the polluted water body, and then the system for determining pollutant control strategy is controlled to execute instructions corresponding to the pollutant control strategy. Then the pollutant control strategy is adjusted in real time to ensure intelligent control of pollutant treatment of the polluted water body. In this solution, the pollutant control strategy for the polluted water body is generated in real time based on the pollutant distribution information and the water flow information of the polluted water body, and the pollutant control strategy is adjusted by monitoring current water body data of the polluted water body in real time, thereby improving the applicability of the intelligent pollutant treatment for different polluted water bodies, and improving the effects of pollutant management and control for different groundwater pollutions.

Optionally, identifying the pollutant distribution information of the polluted water body based on the historical sample water body data and the water body data of different locations, includes: identifying target historical sample water body data corresponding to the water body data of each of the different locations from the historical sample water body data, and using a pollution level of each of the target historical sample water body data as a corresponding pollution level of the water body data of each location; identifying relative position information between the different locations, and generating pollutant distribution information of the polluted water body based on the relative position information and the corresponding pollution levels of the water body data of the different locations.

In this embodiment, the terminal identifies the target historical sample water body data corresponding to the water body data of each location from the historical sample water body data, and uses the pollution level of target historical sample water body data as the pollution level corresponding to the water body data of the corresponding location. Then the terminal identifies the relative position information between all locations, and generates the pollutant distribution information of the polluted water body based on the relative position information and the pollution levels corresponding to the water body data of the locations. The manner of generating the pollutant distribution information is performing a region-images fitting processing for the pollution levels of the different locations to obtain the pollutant distribution information. Specifically, based on the pollution levels of the different locations and the relative position information between the different locations, the terminal identifies the position information of the pollution center point of the polluted water body through a two-dimensional plane image recognition strategy. Then the terminal, by taking the pollution center point as a center of a circle and taking a straight-line distance between each of the different locations and the pollution center point as a radius, constructs a circular range of each of the pollution levels of the polluted water body, thereby obtaining the pollutant distribution information of the polluted water body. The pollutant distribution information is limited by a boundary of the groundwater body, that is, the boundary of the pollutant distribution information includes a boundary of a circular range of each pollution level located in the water flow direction, and a shore boundary located in a direction perpendicular to the water flow direction. The two-dimensional plane image recognition strategy is that the terminal constructs a two-dimensional coordinate system containing each location based on the relative position information between all locations, and then the terminal identifies directions in which pollution levels increase between the locations based on the pollution level of each location. Finally, the terminal performs interpolation calculations based on gradients of pollution levels at different locations to obtain a location having the highest pollution level, and uses position information of the location having the highest pollution level as the position information corresponding to the pollution center point.

Based on the above solutions, the position information corresponding to the pollution center is determined based on the relative position information of each location and the pollution level of each location, and then the pollutant distribution information of the polluted water body is constructed, which improves the accuracy of the constructed pollutant distribution information of the polluted water body.

Optionally, generating the pollutant control strategy for the polluted water body based on the water flow information of the polluted water body and the pollutant distribution information of the polluted water body includes: identifying a hydraulic gradient of the polluted water body and a water flow direction of the polluted water body based on the water flow information; identifying a diffusion direction of the polluted water body and a diffusion rate of the polluted water body, based on the hydraulic gradient of the polluted water body and the water flow direction of the polluted water body and the pollutant distribution information of the polluted water body; dividing the polluted water body into different polluted regions based on the diffusion directions of the polluted water body and the diffusion rates of the polluted water body, obtaining pollution diffusion information corresponding to different polluted regions, and identifying the pollution level corresponding to each polluted region; for each polluted region, based on the pollution level corresponding to each polluted region and the pollution diffusion information corresponding to each polluted region, querying a pollution control sub-strategy corresponding to the polluted region from a pollution-treatment strategy database, and using pollution control sub-strategies for all polluted regions as the pollutant control strategy for the polluted water body.

In this embodiment, the terminal identifies the hydraulic gradient of the polluted water body and the water flow direction of the polluted water body based on the water flow information, and identifies the diffusion directions of the polluted water body and the diffusion rates of the polluted water body, based on the hydraulic gradient of the polluted water body and the water flow direction of the polluted water body and the pollutant distribution information of the polluted water body. The diffusion directions of the polluted water body each are the same as a water flow direction of each region of the polluted water body, and the diffusion rates of the polluted water body are the diffusion rates of different regions in the pollutant distribution information. Then, the terminal divides the polluted water body into different polluted regions based on the diffusion directions of the polluted water body and the diffusion rates of the polluted water body, and obtains the pollution diffusion information corresponding to the different polluted regions. The specific division method is that the terminal regards regions, which have the same diffusion direction, and whose diffusion-rate deviation values each are not greater than a preset deviation threshold, as one polluted region. Finally, the terminal identifies the pollution level corresponding to each polluted region. Each polluted region may include one or more pollution levels corresponding to the polluted regions. When the polluted region includes multiple pollution levels, the terminal uses the highest pollution level among the multiple pollution levels as the pollution level corresponding to the polluted region.

For each polluted region, based on the pollution level corresponding to the polluted region and the pollution diffusion information corresponding to the polluted region, the pollution control sub-strategy corresponding to the polluted region is queried in the pollution-treatment-strategy database, and the pollution control sub-strategies corresponding to all polluted regions are used as the pollutant control strategy for the polluted water body. Each pollution control sub-strategy corresponds to a diffusion rate range and a pollution level range. The terminal identifies a diffusion rate range to which the diffusion rate of each polluted region belongs, and a pollution level range to which a pollution level corresponding to each polluted region belongs, and determines the pollution control sub-strategy corresponding to each polluted region.

Specifically, the process of determining the pollution control sub-strategy is as follows. The terminal determines a comprehensive pumping rate and a comprehensive high-pressure water injection rate of a polluted region based on the diffusion rate, diffusion direction, and pollution level of the polluted region. Then, the terminal identifies the number of extraction wells contained in the polluted region, position information of each extraction well contained in the polluted region, a unit pumping rate of each extraction well, a unit high-pressure water injection rate of each extraction well, and information of a ratio of the number of extraction wells required by the groundwater directional injecting subsystem to the number of vacuum extraction capture wells.

Then, the terminal divides the comprehensive pumping rate of the polluted region by the unit pumping rate of the extraction well to obtain the first number of extraction wells required by the groundwater extracting subsystem in the polluted region, and divides the comprehensive high-pressure water injection rate of the polluted region by the unit high-pressure water injection rate of the extraction well to obtain the second number of extraction wells required by the groundwater directional injecting subsystem in the polluted region. Finally, based on the information of the ratio of the number of the extraction wells required by the groundwater directional injecting subsystem to the number of the vacuum extraction capture wells, the terminal calculates the third number of vacuum extraction capture wells corresponding to the second number.

The terminal selects the first number of extraction wells at the starting end of the diffusion of the polluted region from the extraction wells included in the polluted region and uses them as the pumping wells required by the groundwater extracting subsystem; selects the third number of extraction wells at the starting end of the diffusion of the polluted region and used them as the vacuum extraction capture wells; and selects the second number of extraction wells at the ending end of the diffusion of the polluted region and uses them as the injection wells required by the groundwater directional injecting subsystem in the polluted region. Finally, the terminal uses each pumping well, each injection well, each vacuum extraction capture well, and position information of each well as the pollution control sub-strategy for the polluted region.

Based on the above scheme, after the polluted water body is divided by identifying water flow information, the pollution control sub-strategy of each polluted region is determined, thereby improving effects of pollutant control for different polluted regions.

Optionally, based on the change information of the water body data of each location, adjusting the pollutant control strategy for the polluted water body to obtain a new pollutant control strategy includes following steps. Based on the change information of the water body data of each location, current pollutant distribution information of the polluted water body is identified, and return to execute the step of collecting the water flow information of the polluted water body to obtain a new pollution control sub-strategy corresponding to each new polluted region of the polluted water body, and the terminal uses the new pollution control sub-strategies for all new polluted regions as the new pollutant control strategy for the polluted water body.

In this embodiment, the terminal determines the new water body data of each location based on the change information of the water body data of each location, and identifies the current pollutant distribution information of the polluted water body based on the water body data of each historical sample and the new water body data of each location. Then, the terminal returns to execute the step of collecting the water flow information of the polluted water body, and obtains the new pollution control sub-strategy corresponding to each new polluted region of the polluted water body. Finally, the terminal uses the new pollution control sub-strategies corresponding to all new polluted regions as the new pollutant control strategy for the polluted water body. After the pollutants are extracted, the density of the current polluted water body decreases, so the flow speed of the groundwater to which the current polluted water body belongs will also increase, so it is necessary to re-collect the water flow information of the polluted water body and re-determine each polluted region.

Based on the above solution, by detecting the change information of the water body data of the current polluted water body in real time, the new polluted regions are adjusted, and the new pollution control sub-strategy corresponding to the new polluted regions are obtained, thereby improving the effects of pollutant intelligent manage and control for the polluted water body, thereby improving the management and control efficiency of the polluted water body.

Optionally, iteratively executing the steps of executing the pollutant control strategy and detecting the change information of the water body data of each of the different locations, until the current water body data of each location meet the standard water body condition, includes: for each location, collecting the current water body data of each location, and identifying the first historical sample water body data corresponding to the current water body data of the location from the historical sample water body data, and using the pollution level of the first historical sample water body data as the pollution level corresponding to the current water body data of the location; judging whether there is a pollution level greater than a pollution level threshold; and ending the iterative execution if there is no pollution level greater than the pollution level threshold.

In this embodiment, the terminal collects the current water body data of each location, and identifies the first historical sample water body data corresponding to the current water body data of the location from the historical sample water body data, and uses the pollution level of the first historical sample water body data as the pollution level corresponding to the current water body data of the location. Then, the terminal judges whether there is a pollution level greater than the pollution level threshold which is preset in the terminal. If there is no pollution level greater than the pollution level threshold, the iterative execution ends, and if there is a pollution level greater than the pollution level threshold, the terminal iteratively executes the steps of performing the pollutant control strategy and detecting the change information of the water body data of each of the different locations, until there is no pollution level greater than the pollution level threshold.

Based on the above solution, by determining a termination condition for the iterative execution, an energy waste is avoided, thereby improving the energy optimization effect and intelligence level of the method for determining the pollutant control strategy.

In an embodiment as shown in FIG. 1, a system for determining a pollutant control strategy is provided. The system includes a head pressure detecting subsystem, a groundwater extracting subsystem, a groundwater directional injecting subsystem, a flow detecting subsystem, a pollutant monitoring subsystem, and a control center subsystem. The control center subsystem is connected to the head pressure detecting subsystem, the groundwater extracting subsystem, the groundwater directional injecting subsystem, the flow detecting subsystem, and the pollutant monitoring subsystem respectively. The head pressure detecting subsystem is connected to the groundwater extracting subsystem and the groundwater directional injecting subsystem respectively. The flow detecting subsystem is configured to collect the water flow information of the polluted water body, and transmit the water flow information of the polluted water body to the control center subsystem. The pollutant monitoring subsystem is configured to collect water body data of different locations of the polluted water body, and transmit the water body data of different locations of the polluted water body to the control center subsystem. The groundwater extracting subsystem and the groundwater directional injecting subsystem are configured to receive the pollutant control instructions of executing the pollutant control strategy transmitted by the control center subsystem, and configured to execute the pollutant control strategy according to the pollutant control instructions. The head pressure detecting subsystem is configured to detect the execution information of the groundwater extracting subsystem and the groundwater directional injecting subsystem responding to the pollutant control instructions, as well as the operation information of the groundwater extracting subsystem and the groundwater directional injecting subsystem, and transmit the execution information and the operation information of the groundwater extracting subsystem and the groundwater directional injecting subsystem to the control center subsystem. The control center subsystem is configured to execute the method for determining the pollutant control strategy.

Preferably, the groundwater directional injecting subsystem includes a vacuum extraction subsystem and an injection pump, and the groundwater extracting subsystem includes a suction pump. The injection pump of the groundwater directional injecting subsystem, the suction pump of the groundwater extracting subsystem, and the vacuum extraction subsystem are arranged in the same extraction well. The number of extraction wells is greater than three, and one extraction well cannot perform tasks corresponding to two or more subsystems at the same time. The groundwater directional injecting subsystem is configured to, based on the pollutant control instructions, identify the extraction well corresponding to the injection well and the extraction well corresponding to the vacuum extraction capture well from all extraction wells, and simultaneously start the injection pump in the extraction well corresponding to the injection well and the vacuum pumping device of the extraction well corresponding to the vacuum extraction capture well. The groundwater extracting subsystem is configured to, based on the pollutant control instructions, identify the extraction well corresponding to the pumping well from all extraction wells, and start the suction pump of the extraction well corresponding to the pumping well. The head pressure detecting subsystem includes a water flow pressure sensor and a flow direction sensor. The flow detecting subsystem includes a water flow pressure sensor. The water flow pressure sensor and flow direction sensor of the head pressure detecting subsystem, and the water flow pressure sensor of the flow detecting subsystem are arranged in the same extraction well.

Preferably, the injection pump is configured to inject high-pressure water into the polluted water body, and the vacuum pumping device is configured to perform a vacuum extraction operation on the polluted water body to create a low-pressure zone, and a hydraulic communication channel is constructed based on the low-pressure zone and the high-pressure water to guide the high-pressure water into the polluted water body through the hydraulic communication channel so as to carry out injection operation on the polluted water body. When the extraction well is an injection well, the extraction well opens directing mesh holes arranged therein, and the directing mesh holes are configured to guide the injection directions of the high-pressure water. The hole spacing d between the directing mesh holes is less than 5 cm, and a hole spacing threshold preset by the terminal is 5 cm. The directing mesh holes are wrapped with a protective net outside, and the protective net is configured to prevent pollutants from blocking the directing mesh holes, thereby protecting the operation of the water injection of the high-pressure water. The orientations of the directing mesh holes are determined by the direction of the extraction well corresponding to the vacuum extraction capture well relative to the extraction well corresponding to the injection well. The extraction well includes a first well section exposed to air and a second well section in the water body. Based on the height H₁ of a vadose zone in the first well section and the thickness H₂ of an aquifer corresponding to the second well section, a pressure P of the high-pressure fluid injection of the injection pump and a pumping pressure P′ of the suction pump are determined. The pressure P of the high-pressure fluid injection satisfies >ρg(1.7H₁+H₂), and the pumping pressure P′ forms a drawdown greater than H₂/2.

Specifically, as shown in FIG. 1, the control center subsystem (hereinafter referred to a system) includes a data receiving device, a human-machine interface, a control module, a network server, and a remote login interface. The data receiving device is configured to collect data in real time from the head pressure sensors and water pump flow meters, which are installed at various locations of the pollution plume, and configured to, in combination with the pollutant monitoring system, receive real-time sensor data or laboratory analysis data. The received data are transmitted to the control module, and the control module performs calculations and logical judgments based on the real-time data and the historical data, controlling the extraction pump and the injection pump. The data collected by the data receiving device and the instructions sent by the control module are also transmitted to the human-machine interface (HMI) and the network server in real time, and are stored. The network server may be browsed and operated through a remote login interface. The staff may command the control module through the human-machine interface or remote login interface.

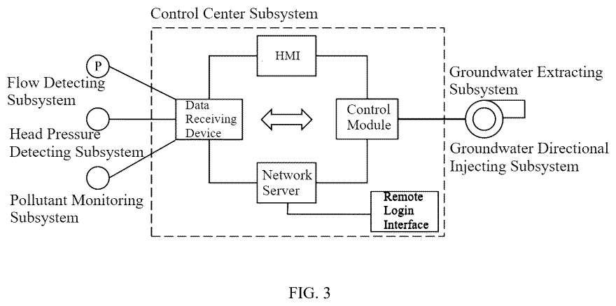

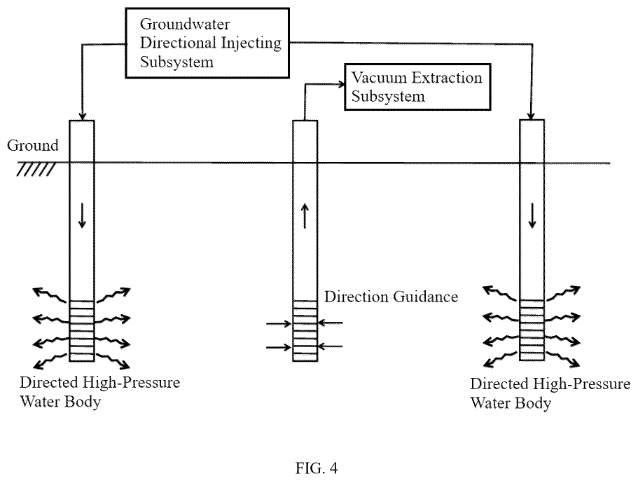

As shown in FIG. 3, the system constructs multiple extraction wells evenly distributed at different locations in the polluted water body, and each extraction well is connected to a pumping pipeline, and then pumping pipelines are connected to the polluted groundwater treatment system through a main pipe. The extraction well includes a head sensor, a suction pump, an injection pump, directing mesh holes, and a protective net. The head sensor includes a water flow pressure sensor and a flow direction sensor, and the water flow pressure sensor is connected to the flow detecting subsystem and the head pressure detecting subsystem respectively, and the flow direction sensor is connected to the head pressure detecting subsystem. All sensors are connected to the control center subsystem through data bus. As shown in FIG. 4, the high-pressure injection subsystem is connected to the vacuum extraction subsystem and the injection pumps through control bus to execute the pollutant control instructions. The groundwater directional injecting subsystem includes two types of injection facilities, some of which are extraction wells, and some of which are water injection ditches. These extraction wells each are equipped with an injection pipeline and an extraction pipeline, and in a process of injecting water, the extraction pipelines of these wells are closed and the injection pipelines thereof are opened. The water injection ditches each are located between adjacent extraction wells or located at downstream locations of the extraction wells. The groundwater directional injecting subsystem includes a water treatment subsystem, and drained water of the water treatment subsystem passes through a deep treatment device for water re-injection. The drain pipe of the water treatment subsystem is connected to the groundwater directional injecting subsystem to provide water materials for the groundwater directional injecting subsystem.

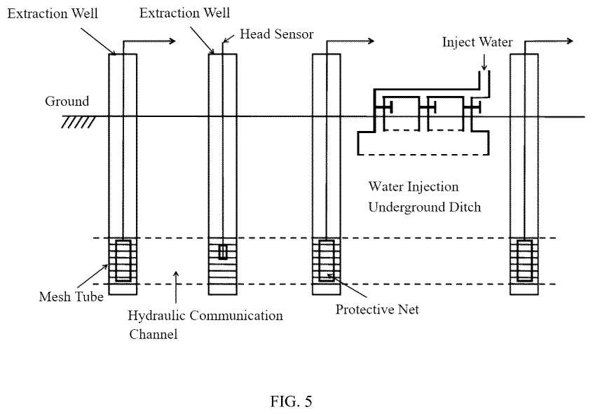

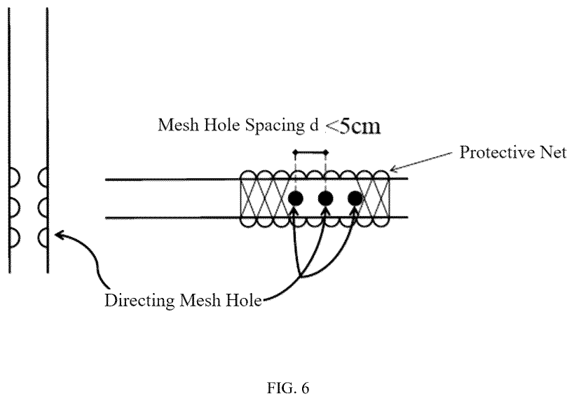

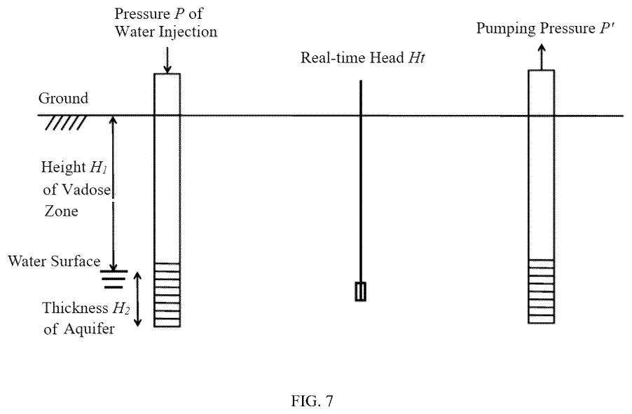

As shown in FIG. 5, the hydraulic communication channel is constructed by the high-pressure water injected by the groundwater directional injecting subsystem, and by the low-pressure zone created by the vacuum extraction capture well in the water extraction process of the groundwater extraction system. The groundwater directional injecting subsystem injects high-pressure fluid through some extraction wells. The main component of the fluid is water, and lubricating materials and pore-filling materials may be added into the fluid according to the needs of the formation structure. The mesh tube of the extraction well corresponding to the high-pressure injection well has directing mesh holes as shown in FIG. 6, and the mesh hole spacing d is less than 5 cm. Some extraction wells are selected from the high-pressure injection wells to serve as the vacuum extraction capture wells, which perform a vacuum extraction while a high-pressure injection is being performed, thus constructing the low-pressure zone to a provide direction guidance for the high-pressure injected fluid. The orientations of the mesh holes of the mesh tube of the high-pressure injection well are determined by the position of the vacuum extraction capture well, thereby enhancing the directionality of the high-pressure injection and ensuring a successful construction of the hydraulic communication channel. As shown in FIG. 7, the extraction well includes the first well section exposed to the air and the second well section in the water body. Based on the height H₁ of the vadose zone in the first well section and the thickness H₂ of the aquifer corresponding to the second well section, the pressure P of the high-pressure water injection of the injection pump and the pumping pressure P′ of the suction pump are determined. The pressure P of the high-pressure water injection satisfies P>ρg(1.7H₁+H₂), and the pumping pressure P′ forms the drawdown greater than H₂/2.

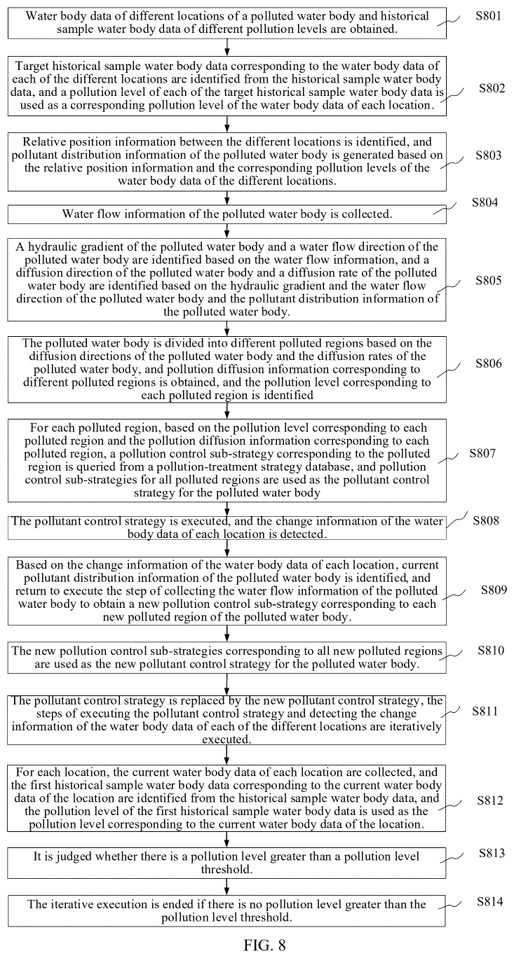

The present application also provides an example of a method for determining a pollutant control strategy. As shown in FIG. 8, the specific determining process includes the following steps S801 to S814.

In Step S801, water body data of different locations of a polluted water body and historical sample water body data of different pollution levels are obtained.

In Step S802, target historical sample water body data corresponding to the water body data of each of the different locations are identified from the historical sample water body data, and a pollution level of each of the target historical sample water body data is used as a corresponding pollution level of the water body data of each location.

In Step S803, relative position information between the different locations is identified, and pollutant distribution information of the polluted water body is generated based on the relative position information and the corresponding pollution levels of the water body data of the different locations.

In Step S804, water flow information of the polluted water body is collected.

In Step S805, a hydraulic gradient of the polluted water body and a water flow direction of the polluted water body are identified based on the water flow information, and a diffusion direction of the polluted water body and a diffusion rate of the polluted water body are identified based on the hydraulic gradient of the polluted water body and the water flow direction of the polluted water body and the pollutant distribution information of the polluted water body.

In Step S806, the polluted water body is divided into different polluted regions based on the diffusion directions of the polluted water body and the diffusion rates of the polluted water body, and pollution diffusion information corresponding to different polluted regions is obtained, and the pollution level corresponding to each polluted region is identified.

In Step S807, for each polluted region, based on the pollution level corresponding to each polluted region and the pollution diffusion information corresponding to each polluted region, a pollution control sub-strategy corresponding to the polluted region is queried from a pollution-treatment strategy database, and pollution control sub-strategies for all polluted regions are used as the pollutant control strategy for the polluted water body.

In Step S808, the pollutant control strategy is executed, and the change information of the water body data of each location is detected.

In Step S809, based on the change information of the water body data of each location, current pollutant distribution information of the polluted water body is identified, and return to execute the step of collecting the water flow information of the polluted water body to obtain a new pollution control sub-strategy corresponding to each new polluted region of the polluted water body.

In Step S810, the new pollution control sub-strategies corresponding to all new polluted regions are used as the new pollutant control strategy for the polluted water body.

In Step S811, the pollutant control strategy is replaced by the new pollutant control strategy, the steps of executing the pollutant control strategy and detecting the change information of the water body data of each of the different locations are iteratively executed.

In Step S812, for each location, the current water body data of each location are collected, and the first historical sample water body data corresponding to the current water body data of the location are identified from the historical sample water body data, and the pollution level of the first historical sample water body data is used as the pollution level corresponding to the current water body data of the location.

In Step S813, it is judged whether there is a pollution level greater than a pollution level threshold.

In Step S814, the iterative execution is ended if there is no pollution level greater than the pollution level threshold.

It should be understood that, although the various steps in the flowcharts involved in various aforementioned embodiments are displayed in sequence as indicated by the arrows, these steps are not necessarily performed in sequence in the order indicated by the arrows. Unless expressly stated herein, the execution of these steps is not strictly restrictive and may be performed in other orders. Moreover, at least part of the steps in the flowcharts involved in various embodiments above may include a plurality of steps or a plurality of stages, which are not necessarily performed at the same moment, but may be executed at different moments, and these steps or stages are not necessarily performed sequentially, but may be performed in turn or alternately with other steps or at least part of the steps or stages of other steps.

Based on the same inventive concept, the embodiment of the present application also provides an apparatus for determining pollutant control strategy for implementing the above-mentioned method for determining pollutant control strategy. The implementation solution provided by the apparatus for solving the problem is similar to the implementation solution described in the above-mentioned method, so for the specific limitations in one or more embodiments of the apparatus for determining pollutant control strategy provided below, reference may be made to the limitations of the method for determining pollutant control strategy above, which will not be repeated here.

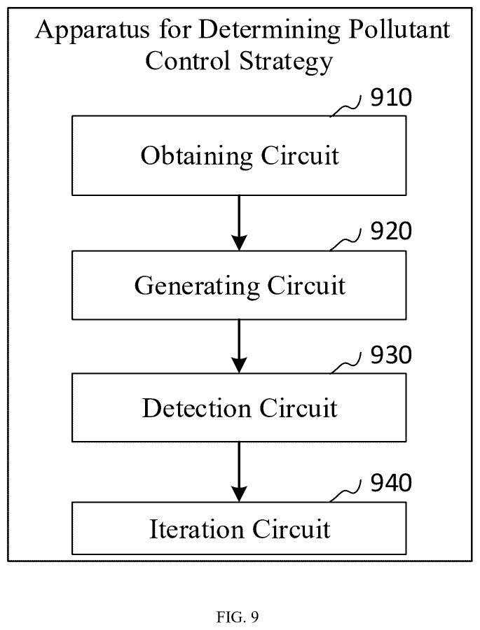

In an embodiment, as shown in FIG. 9, an apparatus for determining a pollutant control strategy is provided, and includes an obtaining circuit 910, a generation circuit 920, a detection circuit 930 and an iteration circuit 940.

The obtaining circuit 910 is configured to obtain water body data of different locations of a polluted water body and historical sample water body data of different pollution levels, and identify pollutant distribution information of the polluted water body based on the historical sample water body data and the water body data of different locations.

The generating circuit 920 is configured to collect water flow information of the polluted water body, and generate a pollutant control strategy for the polluted water body based on the water flow information of the polluted water body and the pollutant distribution information of the polluted water body.

The detection circuit 930 is configured to execute the pollutant control strategy, and detect change information of the water body data of each of the different locations.

The iteration circuit 940 is configured to adjust the pollutant control strategy for the polluted water body based on the change information of the water body data of each location, to obtain a new pollutant control strategy, and configured to replace the pollutant control strategy with the new pollutant control strategy, and configured to iteratively execute the steps of executing the pollutant control strategy and detecting the change information of the water body data of each of the different locations, and configured not to end the iterative execution until the current water body data of each location meet the standard water body condition.

Optionally, the obtaining circuit 910 is specifically configured to identify target historical sample water body data corresponding to the water body data of each location from the historical sample water body data, and use a pollution level of each of target historical sample water body data as the pollution level corresponding to the water body data of each corresponding location, and identify relative position information between the locations, and generate pollutant distribution information of the polluted water body based on the relative position information and the pollution levels corresponding to the water body data of the locations.

Optionally, the generation circuit 920 is specifically configured to: identify a hydraulic gradient of the polluted water body and the water flow direction of the polluted water body based on the water flow information; identify a diffusion direction of the polluted water body and a diffusion rate of the polluted water body, based on the hydraulic gradient of the polluted water body and the water flow direction of the polluted water body and the pollutant distribution information of the polluted water body; divide the polluted water body into different polluted regions based on the diffusion directions of the polluted water body and the diffusion rates of the polluted water body, obtaining pollution diffusion information corresponding to different polluted regions, and identify the pollution level corresponding to each polluted region; for each polluted region, based on the pollution level corresponding to the polluted region and the pollution diffusion information corresponding to the polluted region, query a pollution control sub-strategy corresponding to the polluted region from a pollution-treatment strategy database, and use the pollution control sub-strategies corresponding to all polluted regions as the pollutant control strategy for the polluted water body.

Optionally, the iteration circuit 940 is specifically configured to: identify current pollutant distribution information of the polluted water body based on the change information of the water body data of each location, and return to execute the step of collecting the water flow information of the polluted water body to obtain a new pollution control sub-strategy corresponding to each new polluted region of the polluted water body, and uses the new pollution control sub-strategies corresponding to all new polluted regions as the new pollutant control strategy for the polluted water body.

Optionally, the iteration circuit 940 is specifically configured to: collect the current water body data of each location, and identify the first historical sample water body data corresponding to the current water body data of the location from the historical sample water body data, and use the pollution level of the first historical sample water body data as the pollution level corresponding to the current water body data of the location; judge whether there is a pollution level greater than a pollution level threshold; and end the iterative execution if there is no pollution level greater than the pollution level threshold.

Each circuit and module in the above-mentioned apparatus for determining the pollutant control strategy may be implemented in whole or in part by software, hardware, or combinations thereof. The circuits may be embedded in or independent of the processor of the computer device in a form of hardware, to facilitate the processor calling and executing the operations corresponding to each of the above circuits.

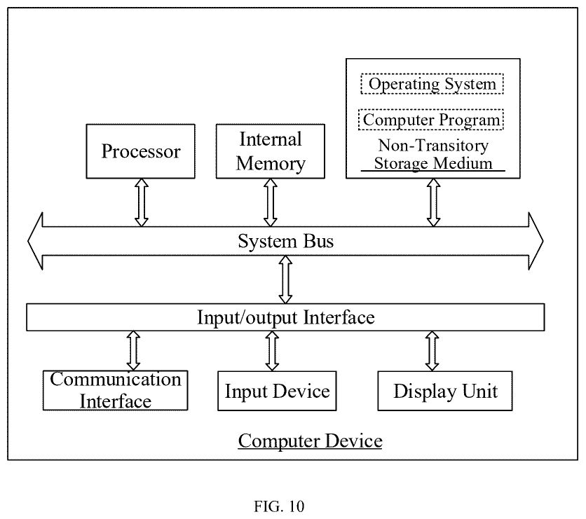

In an embodiment, a computer device is provided, and the computer device may be a terminal, whose internal structure diagram may be shown in FIG. 10. The computer device includes a processor, a memory, a communication interface, a display screen, and an input device, which are connected through a system bus. The processor of the computer device is configured to provide computing and control capabilities. The memory of the computer device includes a non-transitory storage medium and an internal memory. The non-transitory storage medium stores an operating system and computer programs. The internal memory provides an environment for the operation of the operating systems and the computer programs in the non-transitory storage medium. The communication interface of the computer device is configured to communicate with external terminals in a wired or wireless mode, and the wireless communication can be realized by WIFI, mobile cellular network, near field communication (NFC), or other technologies. The computer programs, when being executed by the processor, implement the method for determining the pollutant control strategy. The display screen of the computer device may be an LCD or e-ink display, and the input device of the computer device may be a touch layer covering the display screen, or a key, trackball or trackpad arranged on the housing of the computer device, or an external keyboard, trackpad or mouse, etc.

It should be understood by those skilled in the art that the configuration illustrated in FIG. 10, which is only a block diagram of part of the configuration related to the solution of the present application, does not constitute a limitation on the computer device to which the solution of the present application is applied. The specific computer device may include more or fewer components than those shown in the figure, or may combine some components, or may have a different arrangement of components.

In an embodiment, a computer device is provided, and includes a memory and a processor. The memory has a computer program stored therein, and the processor, when executing the computer program, performs the steps of the method described above.

In an embodiment, a non-transitory computer-readable storage medium is provided, and has a computer program stored thereon. The computer program, when executed by a processor, causes the processor to perform the steps of the method described above.

In an embodiment, a computer program product is provided, and includes executable instructions. The executable instructions, when executed by a processor, cause the processor to perform the steps of the method described above.

It should be noted that the user information (including but not limited to user device information, user personal information, etc.) and data (including but not limited to data used for analysis, stored data, displayed data, etc.) involved in the present application are information and data authorized by the user or fully authorized by all parties.

A person of ordinary skill in the art may understand that implementation of all or part of the processes in the methods of the above embodiments may be completed by instructing the relevant hardware through a computer program. The computer program may be stored in a non-transitory computer-readable storage medium. When the computer program is executed, it may include the processes in the embodiments of the above methods. Any reference to memory, database or other medium used in the embodiments provided in the present application may include at least one of a non-volatile and a volatile memory. The non-volatile memory may include a read-only memory (ROM), a magnetic tape, a floppy disk, a flash memory, an optical memory, a high-density embedded non-volatile memory, a resistive random-access memory (ReRAM), a magnetoresistive random access memory (MRAM), a ferroelectric random-access memory (FRAM), a phase change memory (PCM), or a graphene memory, etc. The volatile memory may include a random-access memory (RAM) or an external cache memory, etc. As an illustration rather than a limitation, the random-access memory may be in various forms, such as a static random-access memory (SRAM) or a dynamic random-access memory (DRAM), etc. The databases involved in the embodiments provided by the present application may include at least one of a relational database and a non-relational database. The non-relational database may include, but is not limited to, a blockchain-based distributed database, etc. The processor involved in the embodiments provided by the present application may be, but is not limited to, a general-purpose processor, a central processor, a graphics processor, a digital signal processor, a programmable logic device, a data processing logic device based on quantum computation, and the like.

The technical features in the above embodiments may be combined arbitrarily. For concise description, not all possible combinations of the technical features in the above embodiments are described. However, provided that they do not conflict with each other, all combinations of the technical features are to be considered to be within the scope described in this specification.

The above-mentioned embodiments only describe several implementations of the present application, and their description is specific and detailed, but should not be understood as a limitation on the patent scope of the present application. It should be noted that, for those skilled in the art variations and improvements may be further made without departing from the conception of the present application, and these variations and improvements all fall within the protection scope of the present application. Therefore, the protection scope of the present application should be subject to the appended claims.

What is claimed is:

1. A method for determining pollutant control strategy, comprising:
obtaining water body data of different locations of a polluted water body and historical sample water body data of different pollution levels, and identifying pollutant distribution information of the polluted water body based on the historical sample water body data and the water body data of the different locations;
collecting water flow information of the polluted water body, and generating a pollutant control strategy for the polluted water body based on the water flow information of the polluted water body and the pollutant distribution information of the polluted water body;
executing the pollutant control strategy, and detecting change information of the water body data of each of the different locations; and
adjusting the pollutant control strategy for the polluted water body based on the change information of the water body data of each of the different locations to obtain a new pollutant control strategy, replacing the pollutant control strategy with the new pollutant control strategy, iteratively executing steps of executing the pollutant control strategy and detecting the change information of the water body data of each of the different locations, until current water body data of each of the different locations meet a standard water body condition.

2. The method according to claim 1, wherein identifying the pollutant distribution information of the polluted water body based on the historical sample water body data and the water body data of the different locations, comprises:
identifying target historical sample water body data corresponding to the water body data of each of the different locations from the historical sample water body data, and using a pollution level of each of the target historical sample water body data as a pollution level of the water body data of each corresponding location; and
identifying relative position information between the different locations, and generating the pollutant distribution information of the polluted water body based on the relative position information and pollution levels of the water body data of the different locations.

3. The method according to claim 1, wherein generating the pollutant control strategy for the polluted water body based on the water flow information of the polluted water body and the pollutant distribution information of the polluted water body, comprises:
identifying a hydraulic gradient of the polluted water body and a water flow direction of the polluted water body based on the water flow information, and identifying a diffusion direction of the polluted water body and a diffusion rate of the polluted water body, based on the hydraulic gradient of the polluted water body and the water flow direction of the polluted water body and the pollutant distribution information of the polluted water body;
dividing the polluted water body into different polluted regions based on identified diffusion directions of the polluted water body and identified diffusion rates of the polluted water body to obtain pollution diffusion information corresponding to different polluted regions, and identifying a pollution level corresponding to each of the different polluted regions; and
querying a pollution control sub-strategy for each of the different polluted regions from a pollution-treatment strategy database, based on the pollution level corresponding to each of the different polluted regions and the pollution diffusion information corresponding to each of the different polluted regions, and using pollution control sub-strategies for the different polluted regions as the pollutant control strategy for the polluted water body.

4. The method according to claim 1, wherein adjusting the pollutant control strategy for the polluted water body based on the change information of the water body data of each of the different locations to obtain a new pollutant control strategy, comprises:
identifying current pollutant distribution information of the polluted water body based on the change information of the water body data of each of the different locations, and returning to execute collecting the water flow information of the polluted water body to obtain a new pollution control sub-strategy corresponding to each new polluted region of the polluted water body; and
using new pollution control sub-strategies for all new polluted regions as the new pollutant control strategy for the polluted water body.

5. The method according to claim 1, wherein iteratively executing the steps of executing the pollutant control strategy and detecting the change information of the water body data of each of the different locations, until the current water body data of each of the different locations meet the standard water body condition, comprises:
collecting current water body data of each of the different locations, and identifying first historical sample water body data corresponding to the current water body data of each of the different locations from the historical sample water body data, and using a pollution level of the first historical sample water body data as a pollution level corresponding to the current water body data of each of the different locations;
judging whether there is a pollution level greater than a pollution level threshold; and
ending iteratively executing the steps of executing the pollutant control strategy and detecting the change information of the water body data of each of the different locations if there is no pollution level greater than the pollution level threshold.

6. The method according to claim 1, wherein the pollutant distribution information of the polluted water body is used to characterize water body regions of the polluted water body corresponding to different pollution levels of the polluted water body.

7. The method according to claim 3, wherein the hydraulic gradient is gradient distribution information of water flow pressures at the different locations.

8. The method according to claim 2, wherein generating the pollutant distribution information of the polluted water body based on the relative position information and the pollution levels of the water body data of the different locations, comprises:
identifying position information of a pollution center point of the polluted water body through a two-dimensional plane image recognition strategy based on the pollution levels of the water body data of the different locations and the relative position information between the different locations; and
constructing a circular range of each of the pollution levels of the water body by taking the pollution center point as a center of a circle and taking a straight-line distance between each of the different locations and the pollution center point as a radius, and obtaining the pollutant distribution information of the polluted water body.

9. The method according to claim 3, wherein dividing the polluted water body into the different polluted regions based on the diffusion directions of the polluted water body and the diffusion rates of the polluted water body, comprises: regarding regions, which have the same diffusion direction, and whose diffusion-rate deviation values each are not greater than a preset deviation threshold, as one polluted region.

10. The method according to claim 9, wherein: each polluted region comprises one or more pollution levels corresponding to the polluted regions; and corresponding to the polluted region comprises multiple pollution levels, the highest pollution level among the multiple pollution levels is used as the pollution level corresponding to the polluted region.

11. The method according to claim 8, wherein: a boundary of the pollutant distribution information of the polluted water body comprises a boundary of a circular range of each pollution level located in a water flow direction, and a shore boundary located in a direction perpendicular to the water flow direction.

12. A system for determining pollutant control strategy, comprising a head pressure detecting subsystem, a groundwater extracting subsystem, a groundwater directional injecting subsystem, a flow detecting subsystem, a pollutant monitoring subsystem, and a control center subsystem, wherein:
the control center subsystem is connected to the head pressure detecting subsystem, the groundwater extracting subsystem, the groundwater directional injecting subsystem, the flow detecting subsystem, and the pollutant monitoring subsystem respectively;
the head pressure detecting subsystem is connected to the groundwater extracting subsystem and the groundwater directional injecting subsystem, respectively;
the flow detecting subsystem is configured to collect water flow information of a polluted water body, and transmit the water flow information of the polluted water body to the control center subsystem;
the pollutant monitoring subsystem is configured to collect water body data of different locations of the polluted water body, and transmit the water body data of the different locations of the polluted water body to the control center subsystem;
the groundwater extracting subsystem and the groundwater directional injecting subsystem are configured to receive pollutant control instructions of executing the pollutant control strategy transmitted by the control center subsystem, and configured to execute the pollutant control strategy according to the pollutant control instructions;
the head pressure detecting subsystem is configured to detect execution information of the groundwater extracting subsystem and the groundwater directional injecting subsystem responding to the pollutant control instructions, and operation information of the groundwater extracting subsystem and the groundwater directional injecting subsystem, and configured to transmit the execution information and the operation information of the groundwater extracting subsystem and the groundwater directional injecting subsystem to the control center subsystem; and
the control center subsystem is configured to execute the method for determining pollutant control strategy of claim 1.

13. The system according to claim 12, wherein the groundwater directional injecting subsystem comprises a vacuum extraction subsystem and an injection pump, and the groundwater extracting subsystem comprises a suction pump, wherein:
the injection pump of the groundwater directional injecting subsystem, the suction pump of the groundwater extracting subsystem, and the vacuum extraction subsystem are arranged in the same extraction well;
the groundwater directional injecting subsystem is configured to identify an extraction well corresponding to an injection well and an extraction well corresponding to a vacuum extraction capture well from all extraction wells based on the pollutant control instructions, and simultaneously start an injection pump in the extraction well corresponding to the injection well and a vacuum pumping device of the extraction well corresponding to the vacuum extraction capture well;
the groundwater extracting subsystem is configured to identify an extraction well corresponding to a pumping well from all extraction wells based on the pollutant control instructions, and start a suction pump of the extraction well corresponding to the pumping well;
the head pressure detecting subsystem comprises a water flow pressure sensor and a flow direction sensor; the flow detecting subsystem comprises a water flow pressure sensor; the water flow pressure sensor and the flow direction sensor of the head pressure detecting subsystem, and the water flow pressure sensor of the flow detecting subsystem are arranged in the same extraction well.

14. The system according to claim 13, wherein:
the injection pump is configured to inject high-pressure water into the polluted water body; the vacuum pumping device is configured to perform a vacuum extraction operation on the polluted water body to create a low-pressure zone; and a hydraulic communication channel is constructed based on the low-pressure zone and the high-pressure water, and is configured to guide the high-pressure water into the polluted water body; or
corresponding to an extraction well being an injection well, the extraction well opens directing mesh holes arranged therein and the directing mesh holes are configured to guide injection directions of the high-pressure water; a hole spacing between the directing mesh holes is less than a preset hole spacing threshold; orientations of the directing mesh holes are determined by a direction of the extraction well corresponding to the vacuum extraction capture well relative to the extraction well corresponding to the injection well; or
each extraction well comprises a first well section exposed to air and a second well section in the water body; based on a height H₁ of a vadose zone in the first well section and a thickness H₂ of an aquifer corresponding to the second well section, a pressure P of a high-pressure water injection of the injection pump and a pumping pressure P′ of the suction pump are determined; the pressure P of the high-pressure water injection satisfies P>ρg(1.7H₁+H₂), and the pumping pressure P′ forms a drawdown greater than H₂/2.

15. The system according to claim 14, wherein:
the preset hole spacing threshold is 5 cm; or
the directing mesh holes are wrapped with a protective net outside.

16. The system according to claim 13, wherein the number of extraction wells is greater than three.

17. An apparatus for determining pollutant control strategy, comprising:
an obtaining circuit configured to obtain water body data of different locations of a polluted water body and historical sample water body data of different pollution levels, and identify pollutant distribution information of the polluted water body based on the historical sample water body data and the water body data of the different locations;
a generating circuit configured to collect water flow information of the polluted water body, and generate a pollutant control strategy for the polluted water body based on the water flow information of the polluted water body and the pollutant distribution information of the polluted water body;
a detection circuit configured to execute the pollutant control strategy, and detect change information of the water body data of each of the different locations;
an iteration circuit configured to adjust the pollutant control strategy for the polluted water body based on the change information of the water body data of each of the different locations to obtain a new pollutant control strategy, configured to replace the pollutant control strategy with the new pollutant control strategy, configured to iteratively execute steps of executing the pollutant control strategy and detecting the change information of the water body data of each of the different locations until current water body data of each of the different locations meet a standard water body condition.

18. A computer device, comprising a processor, and a memory having computer programs stored thereon, wherein the processor, when executing the computer programs, performs steps of the method of claim 1.

19. A non-transitory computer-readable storage medium, having computer programs stored thereon, wherein the computer programs, when executed by a processor, cause the processor to perform steps of the method of claim 1.

20. A computer program product, comprising executable instructions, wherein, the executable instructions, when executed by a processor, cause the processor to perform steps of the method of claim 1.
